(19) United States
(12) Plant Patent Application Publication
(10) Pub. No.: US 2014/0165245 P1
(43) Pub. Date: Jun. 12, 2014

(54) Capsicum annuum PLANT NAMED SLP2B322

(50) ;

(71) Applicant: Syngenta Participations AG, Basel (CH)

(72) Inventor: Binyamin NIR, Shikmim (IL)

(73) Assignee: Syngenta Participations AG, Basel (CH)

(21) Appl. No.: 13/987,940

(22) Filed: Sep. 17, 2013

(30) Foreign Application Priority Data
Dec. 6, 2012 (NL) PPS1433 PVP

(51) Int. Cl. A01H 5/00

(52) U.S. Cl. PLT/258

(57) ABSTRACT

A new Capsicum annuum plant named ‘SLP2B322’ distinguished by its combination of seedless fruits, bright red mature fruit color, fruit diameter of about 3 to 3.5 cm, fruit weight of about 20 to 30 grams, and ability to set fruit under relatively hot conditions.

### CROSS-REFERENCE TO RELATED APPLICATIONS

This application claims the benefit under 35 U.S.C. §119(f) from Netherlands Plant Variety Rights application No. PPS1433, filed Dec. 6, 2012.

### LATIN NAME OF THE GENUS AND SPECIES OF THE PLANT CLAIMED

Capsicum annuum

### VARIETAL DENOMINATION

‘SLP2B322’

### BACKGROUND OF THE NEW PLANT

The present invention comprises a new and distinct cultivar of Capsicum annuum, designated ‘SLP2B322’.

The new C. annuum ‘SLP2B322’ was discovered and selected as a single plant in Gedera, Israel in 2010, from the progeny of a cross between SC09-F4-33-b as the female parent (not patented) and AJ06-F3-378-19 as the male parent (not patented).

The new cultivar was first asexually propagated by root cuttings in Bet Dagan, Israel in February, 2010 and has been asexually propagated since that time by cutting and rooting in Gedera, Israel and Almeria, Spain. The distinctive characteristics of this new C. annuum are stable and reproduce true to type through successive generations of asexual reproduction.

### SUMMARY OF THE INVENTION

‘SLP2B322’ is a distinctive variety of C. annuum characterized by the following traits, which have been repeatedly observed and in combination distinguish ‘SLP2B322’ as a new and distinct C. annuum cultivar:

1. Seedless fruits

2. Bright red mature fruit color

3. Fruit diameter of about 3 to 3.5 cm

4. Fruit weight of about 20 to 30 grams.

5. Ability to set fruit under relatively hot conditions (e.g., 30° C. to 35° C. during the day and 25° C. to 30° C. at night).

Essentially all of the fruits of cultivar ‘SLP2B322’ are completely seedless. However, in cases where ‘SLP2B322’ has been grown in the vicinity of a fertile variety, there are occasional fruits with seeds due to cross-pollination by insects transferring pollen from the fertile variety to ‘SLP2B322’.

### BRIEF DESCRIPTION OF THE DRAWINGS

The accompanying photographs show typical characteristics of the new cultivar.

FIG. 1 shows mature fruits of cultivar ‘SLP2B322’.

FIG. 2 shows plants of cultivar ‘SLP2B322’ growing in a plastic greenhouse in Mivtahime, Israel. This image was taken in December 2012, five and one-half months after planting.

### DETAILED BOTANICAL DESCRIPTION OF THE NEW CULTIVAR

The following is a detailed botanical description of a new and distinct variety of C. annuum known as ‘SLP2B322’. Those skilled in the art will appreciate that certain characteristics will vary with older or younger plants. Further, ‘SLP2B322’ has not been observed under all possible environmental conditions. Where dimensions, sizes, colors and other characteristics are given, it is to be understood that such characteristics are approximations or averages set forth as accurately as practicable. The variety may differ from the descriptions set forth herein with variations in environmental and cultural conditions.

‘SLP2B322’ is able to set fruit throughout the year when grown in a plastic greenhouse in southern Israel, including under cold (November to March) and hot (April to June) temperatures, although the set is reduced in July and August under these conditions.

A technical description of the variety is provided below in Table 1 based upon observations of 5-month-old plants grown under greenhouse conditions in Gedera, Israel during the Fall season.

TABLE 1

| Characteristic | Value |
|---|---|
| Short technical description | Bush-type plant, |
| | medium internodes, |
| | produces seedless fruit, |
| | good ability to |
| | set in relatively hot |
| | conditions |
| Method of reproduction | vegetatively propagated |
| Plant: length of stem | short-medium |
| Plant: shortened internodes | absent (indeterminant) |
| (in upper part) | |
| Plant: number of internodes between | none |
| 1st flower and shortened internodes | |
| (Varieties with shortened internodes only) | |
| Plant: length of internodes (on primary side | medium |
| shoots)(varieties without shortened internodes) | |
| Plant: anthocyanin coloration of nodes | present |
| Stem: Intensity of anthocyanin | medium-strong |
| coloration of nodes | |
| Stem: hairiness of nodes | weak |
| Plant: vigor | strong |
| Plant: height | compact |
| Leaf: length of blade | medium |
| Leaf: intensity of green colour | medium |
| Leaf: shape | ovate |
| Leaf: blistering | weak |
| Leaf: profile in cross section | moderately concave |
| Leaf: glossiness | very weak-weak |
| Leaf: Undulation of margin | medium |
| Leaf: width of blade | medium |
| Flower: anthocyanin coloration in anther | absent |
| Peduncle: attitude | semi-drooping |
| Time of beginning of flowering (1st | medium |
| flower on 2nd flowering node) | |
| Fruit: colour (before maturity) | green |
| Fruit: intensity of colour | medium |
| (before maturity) | |
| Fruit: anthocyanin coloration | absent |
| (before maturity) | |
| Fruit: general impression | conical |
| Fruit: attitude | horizontal |
| Fruit: length | short-medium |
| Fruit: diameter | small |
| Fruit: ratio length-diameter | medium |
| Fruit: shape in longitudinal section | triangular |
| Fruit: shape in cross section | circular |
| (at level of placenta) | |
| Fruit: sinuation of pericarp at basal part | very weak |
| Fruit: sinuation of pericarp excluding | absent of very weak |
| basal part | |
| Fruit: texture of surface | smooth or very slightly |
| | wrinkled |
| Fruit: colour (at maturity) | red |
| Fruit: intensity of colour (at maturity) | medium-dark |
| Fruit: glossiness | strong |
| Fruit: stalk cavity | absent |
| Fruit: depth of stalk cavity | very shallow |
| Fruit: shape of apex (PBR) | very acute |
| Fruit: depth of interloculary grooves | very shallow |
| Fruit: number of locules | predominantly two |
| Fruit: length (cm) | 8 to 12 |
| Fruit: diameter (cm) | 3 to 3.5 |
| Fruit: thickness of flesh | thin |
| Stalk: length | medium |
| Fruit: capsaicin in placenta | absent |
| Stalk: thickness | medium |
| Calyx: aspect | non enveloping |
| Seed | Absent |
| Time of maturity | medium |
| Ripening can be compared with: | KAPPY |
| Potato Virus Y pathotype 0 | S |
| Potato Virus Y pathotype 1 | Unknown |
| Potato Virus Y pathotype 2 | Unknown |
| Tobamovirus pathotype P0 | S |
| Tobamovirus pathotype P0, P1 | S |
| Tobamovirus pathotype P0, P1, P1-2 | S |
| Tobamovirus pathotype P0, P1, P1-2, P1-2-3 | S |
| Tomato Spotted Wilt Virus | S |

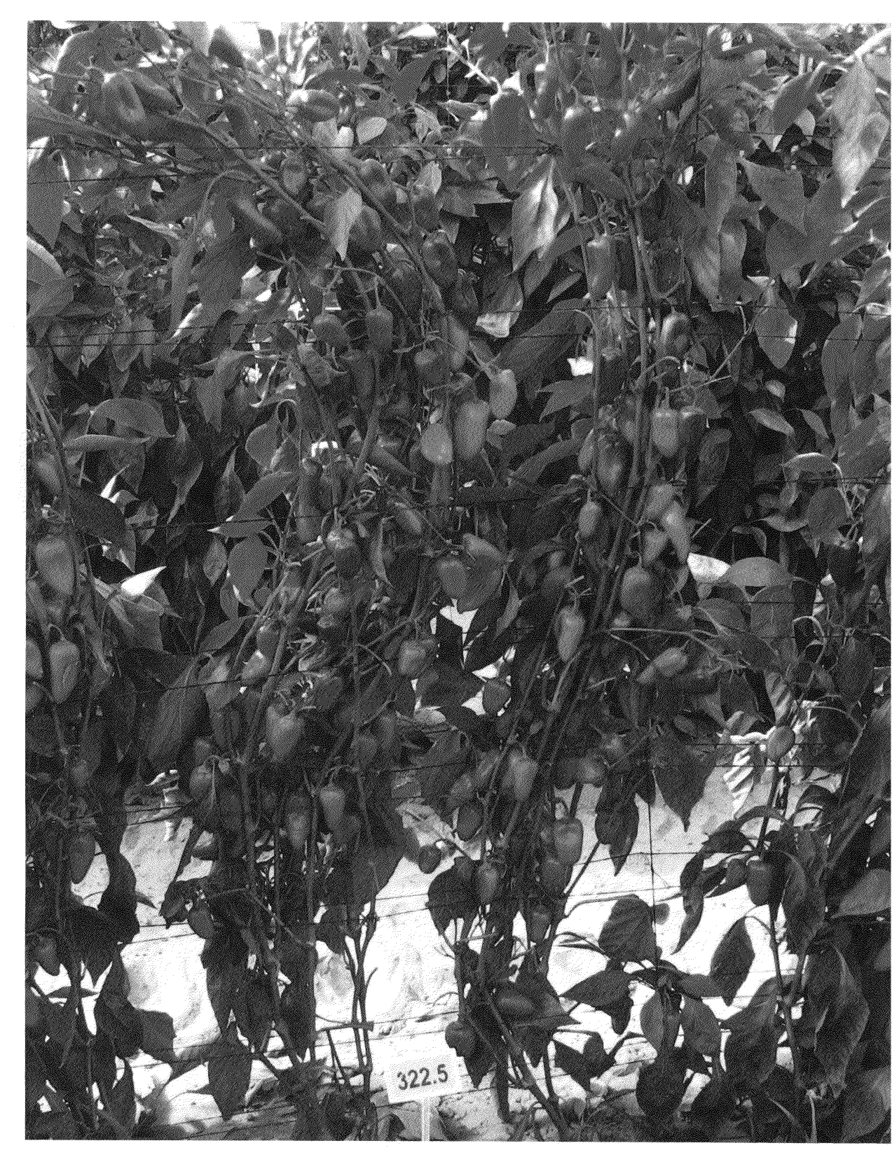

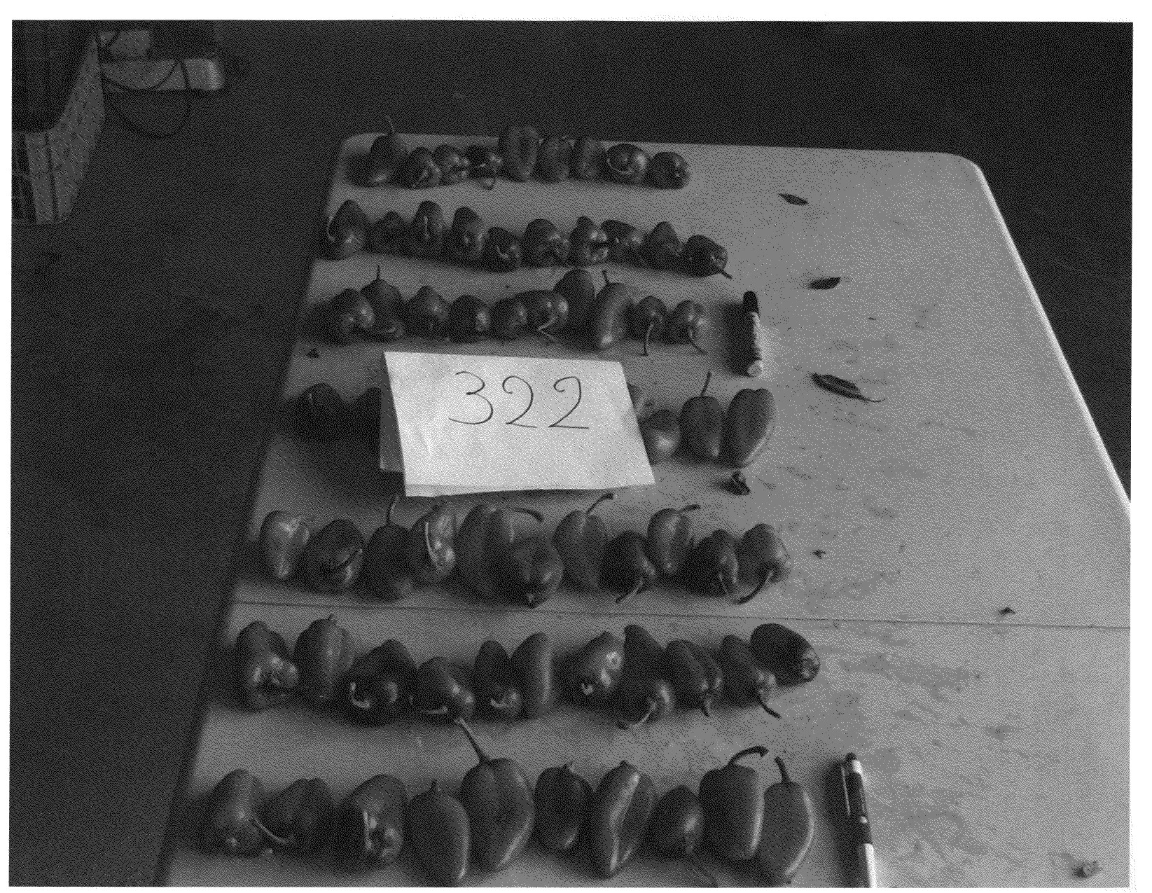

These and other features and characteristics of cultivar ‘SLP2B322’ are apparent from FIGS. 1 and 2.

### Comparisons with Other C. annuum Cultivars

‘SLP2B322’ is most similar to commercial C. annuum cultivar ‘SLP2B131’, which is also a seedless variety. As shown in Table 2, ‘SLP2B322’ can be distinguished from cultivar ‘SLP2B131’ at least with respect to fruit diameter, fruit weight, mature fruit colour, and season for fruit setting.

TABLE 2
SLP2B322 has in comparison with:

| | CHARACTER- | | |
|---|---|---|---|
| | ISTIC | | |
| DENOMINA- | IN WHICH THE | | |
| TION | COMPARISON | STATE OF | |
| OF | VARIETY IS | EXPRESSION OF | STATE OF |
| COMPARISON | DIFFERENT | COMPARISON | EXPRESSION |
| VARIETY | FROM SLP2B322 | VARIETY | OF SLB2B322 |
| SLP2B131 | Fruit diameter | 2-3 cm | 3-3.5 cm |
| SLP2B131 | Fruit weight | 20 to 30 g | 10 to 20 g |
| SLP2B131 | Mature fruit | Dark red | Bright red |
| | intensity | | |
| SLP2B131 | Fruit setting season | Late (moderate | Early (Can set |
| | | temperatures | even in high |
| | | needed for good | temperatures) |
| | | set) | |

What is claimed is:

1. A new and distinct variety of Capsicum annuum plant named ‘SLP2B322’, substantially as described and illustrated herein.
